(19) United States
(12) Patent Application Publication
(10) Pub. No.: US 2007/0169775 A1
(43) Pub. Date: Jul. 26, 2007

(54) Mechanism for the draft of a high frequency atomization device

(75) Inventors: Wen-Pin Chen, Taipei (TW); Nai-Ying Jean, Taipei (TW)

Correspondence Address: Troxell Law Office Pllc;Suite 1404, 5205 Leesburg Pike, Falls Church, VA, 22041, US

(73) Assignee: Kai Chih Industrial Co., Ltd.

(21) Appl. No.: 11/335,549

(22) Filed: Jan. 20, 2006

(51) Int. Cl. A61M 11/00; B05B 17/06

(52) U.S. Cl. 128/200.16; 128/200.14

(57) ABSTRACT

A mechanism for the draft of a high frequency atomization device, which has particular application to supporting a cantilever excitation device on the surface of a large amount of operating liquid using a floating support method, thereby enabling a vibratable plate to accurately position on the liquid surface of any height and bring into effect quantitative power. The excitation device is structured from a block piezoelectric ceramic actuator and the vibratable plate, which extends from one side of the actuator and joined thereto using a cantilever method. The excitation device floats on the liquid surface of the operating liquid using a floating support. An operating side of a free end of the vibratable plate maintains a definite directed amount of effect on the liquid surface, and is able to acquire comparable load conditions and bring into effect quantitative power.

### BACKGROUND OF THE INVENTION

(a) Field of the Invention

The present invention relates to a mechanism for the draft of a high frequency atomization device, which has particular application to supporting an excitation device on the surface of a large amount of operating liquid using a floating support method. After supporting the excitation device, the vibratable plate is able to acquire comparable load conditions and bring into effect quantitative power.

(b) Description of the Prior Art

A conventional liquid atomization device primarily uses high frequency vibrating equipment immersed in an aqueous liquid to excite vibrating energy waves that break up the liquid surface to from a mist, or a vibrating equipment, interior of which is joined to and actuates a vibratable plate, is used to cause wave motion kinetic energy excitation of the aqueous liquid.

If a disk-type piezoelectric ceramic is positioned beneath the liquid surface, after supplying electricity, energy waves produced from the high frequency vibration are used to impact the liquid surface, thereby breaking down the cohesive tension of the liquid surface and atomizing the liquid. Because each of the aforementioned vibrating actuators is positioned within the liquid, thus, the largest portion of the kinetic energy is assimilated by the liquid and wasted.

Referring to FIG. 1, which shows a design for an atomization and excitation device introduced by S. C. Johnson & Son Inc., in recent years, wherein an atomization and excitation device 1 is structured to include a disk type piezoelectric ceramic actuator 100, in which a through hole 101 is formed, and a circular vibratable plate 102 joined to a side of the piezoelectric ceramic actuator 100. A hemispherical surface 103 is formed to protrude from a center portion of the circular vibratable plate 102, and a plurality of vibratable holes 104 are densely distributed in the hemispherical surface 103. The atomization and excitation device 1 acquires liquid from a liquid source by using a liquid guide fiber 105, one end of which extends into a container 106, and the liquid guide fiber 105 is used to draw up liquid contained within the container 106. Moreover, a liquid film formed from surface tension at a top end of the liquid guide fiber 105 is able to come in close contact with the hemispherical surface 103. After the actuator 100 actuates the vibratable plate 102, the vibratable holes 104 produce a vibrational effect that breaks up the liquid to form a mist. Such a configuration is applicable for implementation with the container 106 filled with a small amount of liquid.

Height of the liquid surface within the container 106 produces a change in liquid guide efficiency of the liquid guide fiber 105. Hence, design of the liquid guide fiber 105 affects efficiency of its capillarity effect, and results in a nonuniform amount of atomization and excitation formed.

Moreover, regarding the design of the liquid guide fiber 105, if the liquid contained within the container 106 has medicinal properties and is mixed with medicinal substances, which are in liquid state or powder form, and if the specific gravity of the substances differs from that of the liquid, then the substances will either float or sink in the liquid, and drawing up of the liquid by the liquid guide fiber 105 and excitation will cause the excited mist to carry a nonuniform amount of medicinal value.

Furthermore, the capillarity phenomenon of the liquid guide fiber 105 produces a filter effect that further results in the excited mist carrying an insufficient amount of medicinal value.

Poor affinity between the medicinal substances and the liquid filled in the container 106 results in a static state within the container 106 that results in the inability to produce a mixing effect between the medicinal substances and the liquid solution, thereby causing the liquid drawn up by the liquid guide fiber 105 to be separated from the medicinal substances.

The mist excited by the excitation device is generally used for medicinal purposes.

### SUMMARY OF THE INVENTION

In light of the aforementioned shortcomings, the present invention uses a piezoelectric ceramic actuator that is cantilever connected to a vibratable plate to expose the vibratable plate. A free end of the vibratable plate is submerged beneath a liquid surface at an operating position, and the entire structure floats on the liquid surface of an operating liquid using a floating support. The vibrational waves that are produced directly act on the liquid surface, and a portion of the energy is transmitted to the liquid to produce a mixing effect. The vibratable plate maintains a definite directed amount of effect on the liquid surface, and is able to acquire comparable load conditions and bring into effect quantitative power.

To enable a further understanding of said objectives and the technological methods of the invention herein, brief description of the drawings is provided below followed by detailed description of the preferred embodiments.

### BRIEF DESCRIPTION OF THE DRAWINGS

FIG. 1 shows a schematic view depicting positional relationship between a liquid guide fiber and corresponding vibratable plate of a conventional atomization and excitation device.

FIG. 2 shows a basic schematic view depicting structure of an excitation device according to the present invention.

FIG. 3 shows a schematic view depicting an embodiment of the present invention in use.

FIG. 4 shows a schematic view depicting a change-direction member joined between an actuator and a vibratable plate according to the present invention.

FIG. 5 shows a schematic view depicting a bent configuration between the actuator and the vibratable plate according to the present invention.

FIG. 6 shows a schematic view depicting the excitation device obliquely joined to a floating support according to the present invention.

FIG. 7 shows a side view of FIG. 6.

FIG. 8 shows a schematic view of the floating support formed as a circular frame according to the present invention.

FIG. 9 shows a schematic view of the floating support formed as a square frame according to the present invention.

FIG. 10 shows a side view of the frame-shaped floating support according to the present invention.

FIG. 11 shows a schematic view of the actuator laterally joined to the vibratable plate according to the present invention.

FIG. 12 shows a schematic view depicting a circular disk shaped actuator joined to the vibratable plate according to the present invention.

FIG. 13 shows a schematic view depicting the vibratable plate joined to two sides of the circular disk shaped actuator according to the present invention.

FIG. 14 shows a side schematic view of an embodiment of the floating support and the vibratable plate joined to two sides of the actuator according to the present invention.

FIG. 15 shows a schematic view depicting a bent configuration of the vibratable plate joined to two sides of the actuator according to the present invention.

FIG. 16 shows a schematic view depicting a floating support unit joined to a slide track of a limit device through a mount according to the present invention.

FIG. 17 shows a side view of FIG. 16.

FIG. 18 depicts a system of forces of FIG. 17.

FIG. 19 shows a side schematic view of an embodiment of FIG. 16 in a container according to the present invention.

FIG. 20 shows a side schematic view of the limit device further configured with a swing arm according to the present invention.

FIG. 21 shows a schematic view of the limit device further configured with slide columns according to the present invention.

FIG. 22 shows a schematic view of a side of the floating support unit disposed on the limit device by way of the slide columns according to the present invention.

FIG. 23 shows a schematic view depicting vibratable holes formed as linear slots in the vibratable plate according to the present invention.

FIG. 24 shows a schematic view depicting the vibratable holes formed as waveform slots in the vibratable plate according to the present invention.

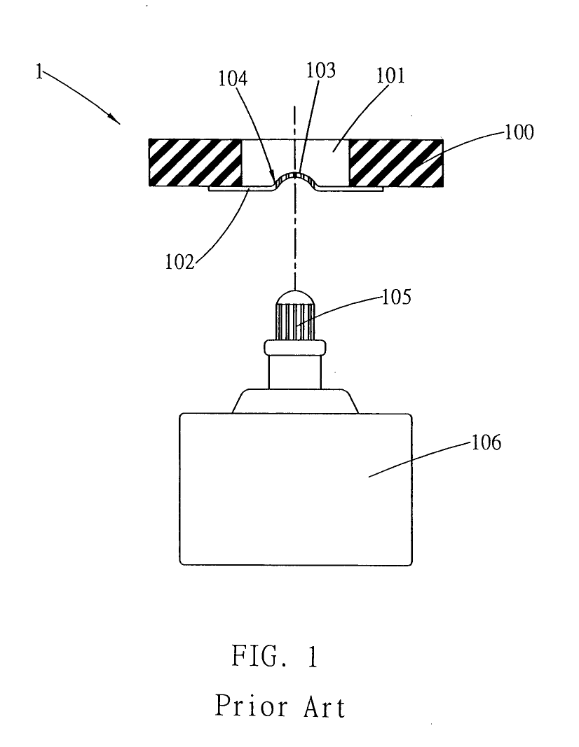

### DETAILED DESCRIPTION OF THE PREFERRED EMBODIMENTS

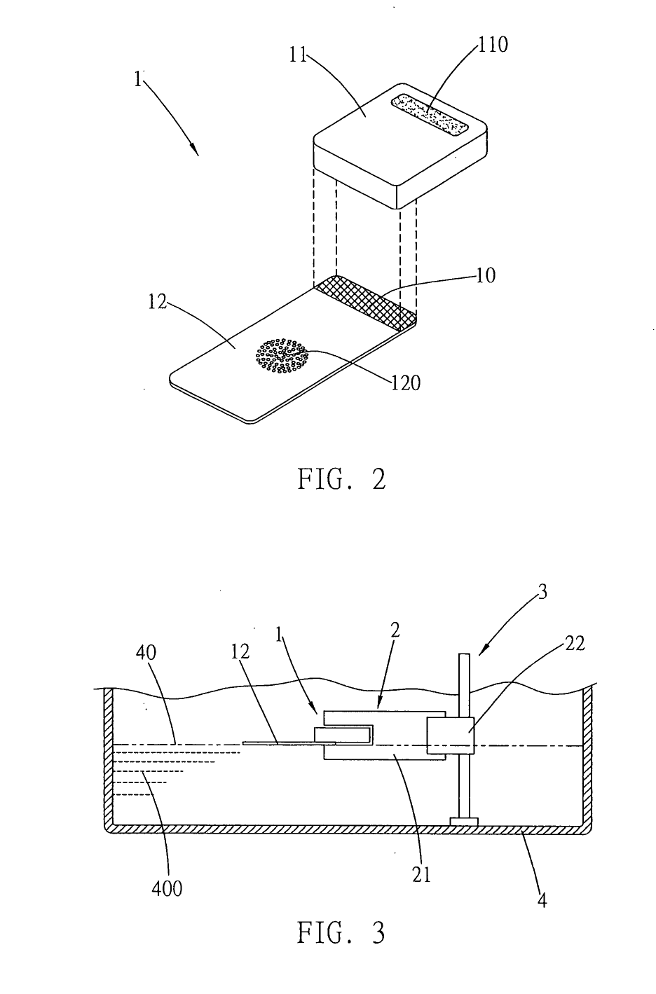

Referring to FIG. 2, which shows an embodiment of an excitation device 1 of the present invention, primarily structured to comprise a vibratable plate 12 joined to a side of a block piezoelectric ceramic actuator 11 using a cantilever method. A joining surface 10 formed on one side of the vibratable plate 11 is used to join to a corresponding end of an underside of the actuator 11. The joining surface 10 can be joined to the actuator 11 using any mechanical or hardware component or agglutination or soldering method.

Vibratable holes 120 are defined in a breadth of the vibratable plate 12. The vibratable holes 120 are minute circular holes that are densely assembled to form a distributed geometric area. Height position of the vibratable holes 120 is such to be adjacent to a surface of a liquid.

An exterior surface of the actuator 11 is coated with a dielectric coating 110 that enables electrical connection to be established with a power cable.

Referring to FIG. 3, which shows the excitation device 1 joined to a floating support 21 that is affixed to a floating support unit 2 forming a horizontal configuration. The floating support 21 floats on a liquid surface 40 of an operating liquid 400 filled in a container 4, thereby enabling the vibratable plate 12 of the excitation device 1 to be in a horizontal position and adjacent to the liquid surface 40.

The floating support unit 2 is joined to a mount 22 that is disposed so as to slide on a limit device 3, thus, height of the floating support unit 2 is subject to disposition of the mount 22 on the limit device 3, thereby forming a vertical displacement utility that enables the floating support unit 2 to be vertically displaced within the container 4.

After power actuates the excitation device 1, the vibratable plate 12 vibrates at high frequency that acts on the liquid surface 40 and causes a liquid film on the liquid surface to break up, thereby producing a vibrationally excited mist with pressure.

A large portion of the kinetic energy of the vibratable plate 12 acts on the liquid surface 40, and a portion of the kinetic energy is transmitted to the operating liquid 400 that causes a mixing or turbulent flow effect in the operating liquid 400.

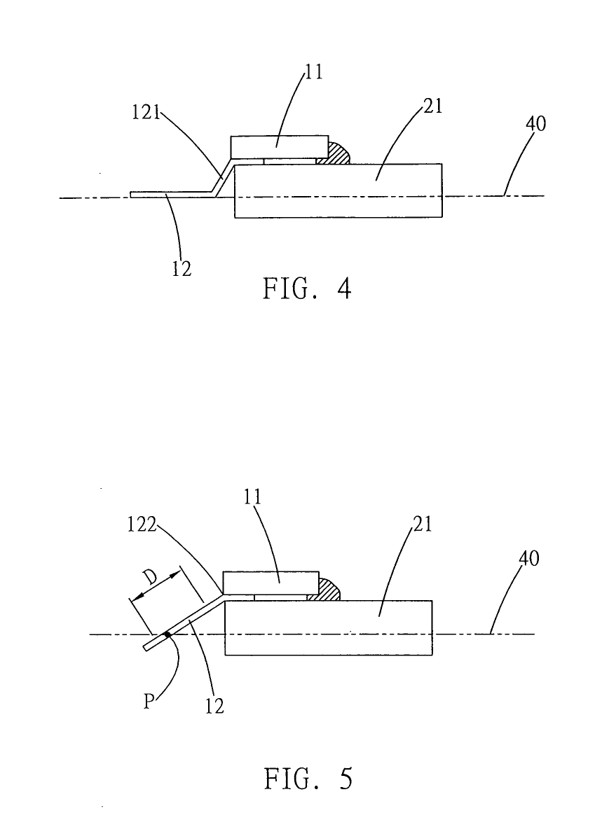

Referring to FIG. 4, any method can be used to join the floating support 21 to the actuator 11, and adjustment of a change-direction member 121 can be used to alter the horizontal and relative height between the vibratable plate 12 and the actuator 11.

Moreover, because height of the floating support above the liquid surface 40 varies according to the mass and density of the floating support 21, thus, adjustment of the change-direction member 121 can be used to enable positioning of the vibratable plate 12 on the liquid surface 40.

Floating height of the floating support 21 relative to the liquid surface 40 can also vary depending on the specific gravity of the operating liquid 400, thus, adjustment of the change-direction member 121 can be similarly used to alter the floating height and ensure that the vibratable plate 12 is horizontally positioned on the liquid surface 40.

Existence of the change-direction member 121 enables disposing the actuator 11 atop the floating support 12, and avoid having to immerse the actuator 11 in the operating liquid 400, thereby preventing possible chemical change that would affect structural binding force, and so on, of the configuration.

Referring to FIG. 5, which shows the actuator 11 joined to a top portion of the floating support 21, and the cantilever extended vibratable plate 12 of the actuator 11 is made to form an oblique angle relative to the floating support 21 by bending at a bent portion 122, thereby enabling the vibratable plate 12 to obliquely break the liquid surface 40 and allow a free end of the vibratable plate 12 to become immersed in the operating liquid 400.

Implementation of the bent portion 122 can similarly ensure that the actuator 11 is not constantly submerged in the operating liquid 400.

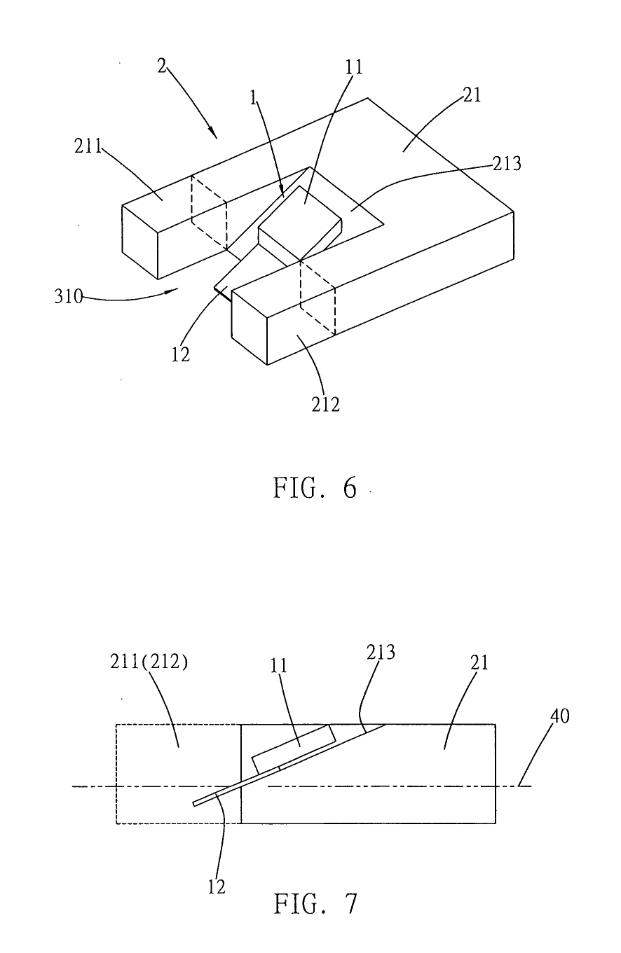

Referring to FIG. 6, which shows the actuator 11 joined to the floating support 21, wherein an opening 210 is formed in one side of the floating support 21. The opening 210 is formed with an oblique side 213 that enables the actuator 11 to be positioned thereon. The vibratable plate 12 is joined to the actuator 11 so as to lie along the same plane surface of the oblique side 213. Hence, disposition of the actuator 11 depends on the angle of the oblique side 213, which correspondingly affects the oblique angle of the vibratable plate 12.

Corners 211, 212 are respectively formed on two sides of the opening 210, and are used to equilibrate the floating support 21, and can further protect the vibratable plate 12 disposed therebetween.

Referring to FIG. 7, the oblique disposition relationship between the vibratable plate 12 and the oblique side 213 is shown, and further depicts the free end of the vibratable plate 12 submerged beneath the liquid surface 40 and the lateral protection of the vibratable plate 12 by the corners 211, 212.

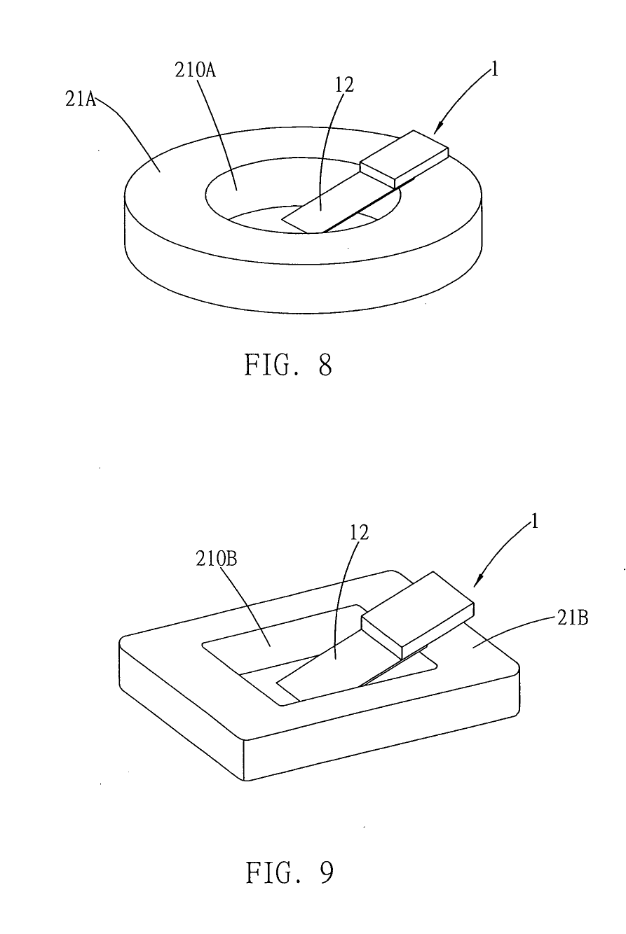

Referring to FIG. 8, the floating support 21 can be formed as a circular frame floating support 21A, an internal through hole 210A of which enables the excitation device 1 to be placed therein and be joined to the circular frame floating support 21. The vibratable plate 12 is obliquely disposed in the through hole 210A, and a periphery of the circular frame floating support 21A is used to thoroughly protect the vibratable plate 12.

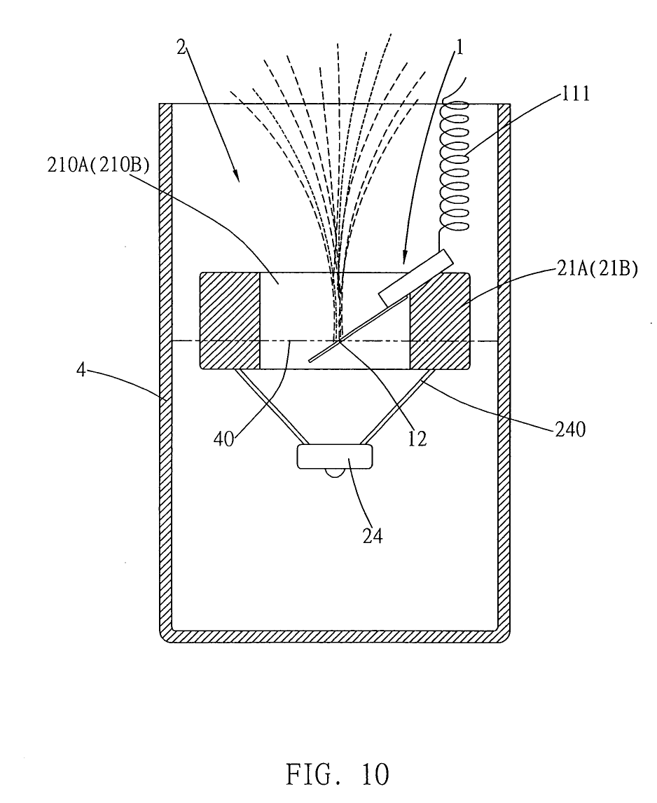

Referring to FIG. 9, which shows the floating support 21 formed as a square frame floating support 21B. An internal through hole 210B of the square frame floating support 21B similarly enables the excitation device 1 to be placed therein and joined to the square frame floating support 21B. The vibratable plate 12 is obliquely disposed in the through hole 210B, and a periphery of the square frame floating support 21B can be similarly used to thoroughly protect the vibratable plate 12.

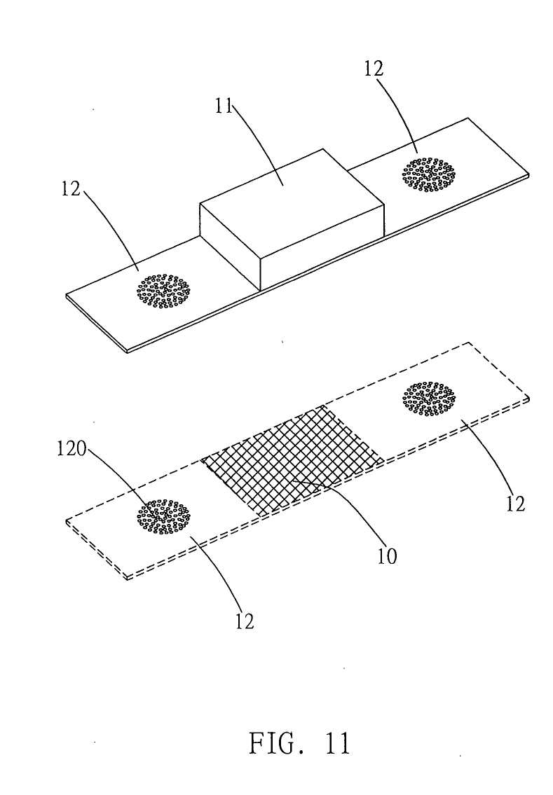

Referring to FIG. 10, an inner surface of the container 4 is symmetrized with respect to the external form of the floating support 21A (21B) according to the structures of the floating support 21A (21B) as depicted in FIGS. 8 and 9 respectively. An inner cross-section of the container 4 is relatively larger to that of the floating support 21A (21B), thereby enabling free movement of the floating support 21A (21B) within the container 4. Moreover, the internal through hole 210A (210B) enables the vibratable plate 12 of the excitation device 1 to be obliquely disposed therein and be submerged beneath the liquid surface 40. The excitation device 1 is connected to a flexible power cable 111 that enables the structured floating support unit 2 to freely rise and descend within the container 4.

A balance weight 24 can be joined to a bottom portion of the floating support 21A (21B). Any method can be used to join the balance weight 24 to the bottom portion of the floating support 21A (21B) or can be joined using connecting cables 240. The balance weight 24 is used to adjust center-of-gravity position of the entire structure, thereby enabling the floating support 21A (21B) to maintain a horizontal disposition as it floats on the liquid surface 40.

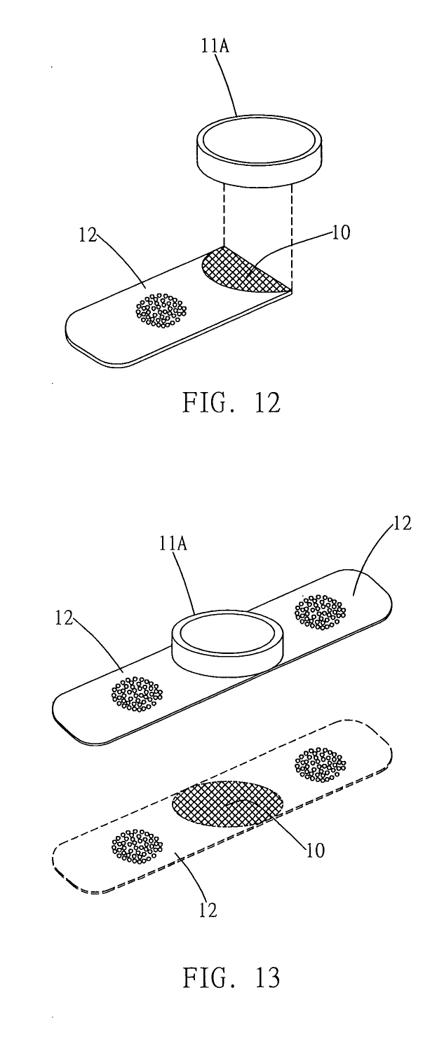

Referring to FIG. 11, which shows the actuator 11 joined to the vibratable plate 12 using a cantilever method, and which is further configured so that two sides of the actuator 11 are respectively symmetrically connected to two vibratable plates 12, thereby achieving a symmetrical configuration. The two vibratable plates 12 are simultaneously actuated by the actuator 11, thereby enabling the two simultaneously vibrating vibratable plates 12 to excite a substantially larger amount of mist by increasing the power of the actuator 11.

The vibratable plates 12 joined to the actuator 11 can be further formed as a strip-form single body, two ends of which are respectively defined with the vibratable holes 120. A joining surface 10 of a central portion of the strip-form single vibratable plate 12, having an area approximately equal to that of a bottom surface of the actuator 11, is joined to the bottom surface of the actuator 11, thereby enabling the vibratable plate 12 and the actuator 11 to form a single integrated body.

Referring to FIG. 12, which shows an actuator configured as a circular disk shaped actuator 11A, one side of which is similarly joined to the vibratable plate 12. The joining surface 10 having an arc-shaped area is formed at one end of the vibratable plate 12, and any method can be used to join the arc-shaped joining surface 10 to the circular disk shaped actuator 11A.

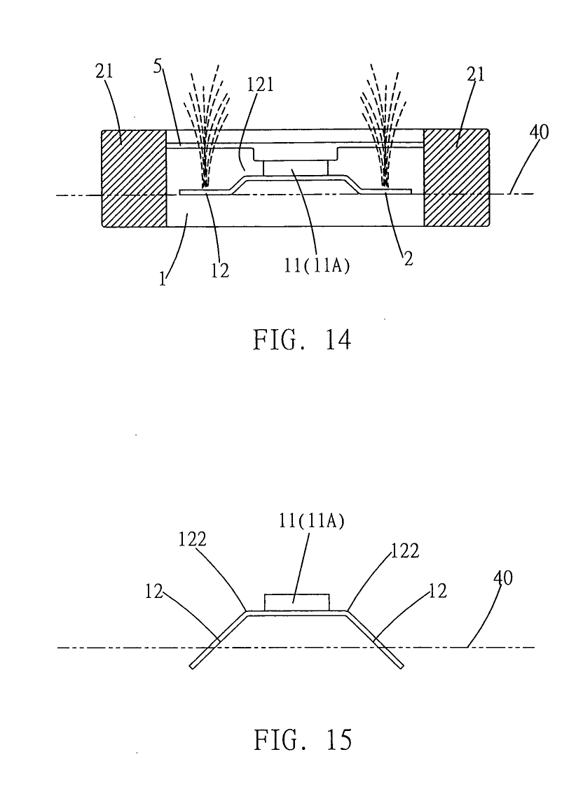

Referring to FIG. 13, which shows the circular disk shaped actuator 11A joined to the vibratable plates 12 using a lateral extended cantilever method whereby a symmetrical method is adopted to join the vibratable plates 12 to the circular disk shaped actuator 11A. The vibratable plates 12 are joined and symmetrically extend from two sides of the actuator 11, thereby enabling the actuator 11 to simultaneously vibrate two symmetrical vibratable plates 12, which can result in exciting a substantially larger amount of mist by supplying the actuator 11 with permitted power or increased power. The vibratable plates 12 can be two independent strips or connected to form a strip-form single body. The joining surface 10 having the same shape as that of the bottom surface of the circular disk shaped actuator 11 is used to join the vibratable plate 12 to the actuator 11, thereby forming a single integrative join that strengthens mechanical capacity of the configuration.

Referring to FIG. 14, the excitation device 1 structured according to that depicted in FIGS, 11, 12 and 13 can be suspended or hung from a beam 5, and joined to a central portion of the floating support 21. The floating support 21 can be formed as one of the aforementioned frame shapes illustrated in FIGS. 8 or 9 or as two floating supports, and symmetrically joined to two ends of the beam 5 to form a balanced floating configuration.

The excitation device 1 is suspended on the beam 5, and the vibratable plate 12 forms effective close contact with the liquid surface 40. Moreover, the vibratable plate 12 joined to the actuator 11A indirectly supports the floating support 21 through the beam 5 and a floating buoyant effect that maintains a definite relative height between the floating support 21 and the liquid surface 40, thereby ensuring that the vibratable plate 12 is effectively positioned on the liquid surface 40.

The change-direction members 121 attached to the vibratable plate 12 can be used to adjust the horizontal disposition and relative height between the vibratable plate 12 and the actuator 11 (11A), thereby enabling the vibratable plate 12 to come in horizontal close contact with the liquid surface 40.

Referring to FIG. 15, the vibratable plate 12 connected to the actuator 11, 11A of FIG. 14 is obliquely submerged beneath the liquid surface 40 using functionality of the bent portions 122.

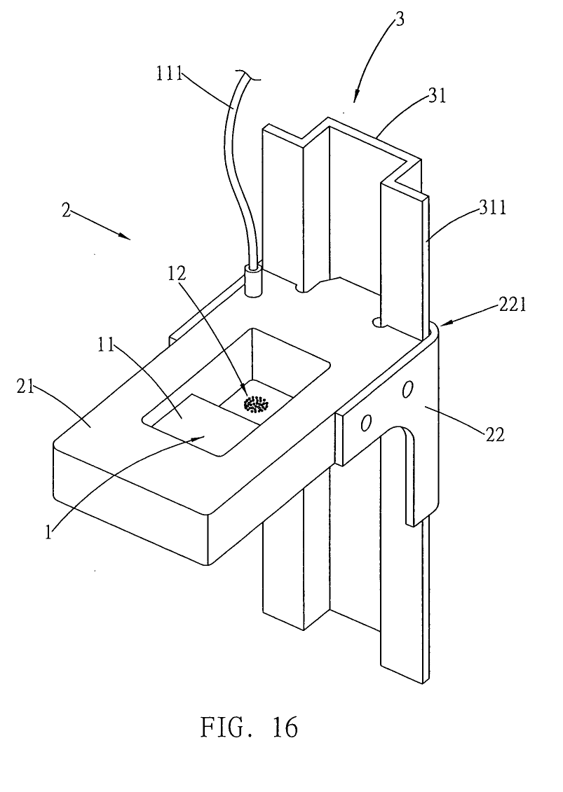

Referring to FIG. 16, the floating support unit 2 primarily uses the floating support 21 to support the excitation device 1. The actuator 11 connected to the excitation device 1 is joined to the vibratable plate 12 using a cantilever method. One end of the floating support 21 is disposed so as to slide on the limit device 3 by means of the mount 22 whereby slide holes 221 are defined in the mount 22, and rails 311 respectively formed on two sides of a slide track 31 enable the mount 22 to slide on the slide track 31 through the slide holes 221 having same shape as that of the rails 311.

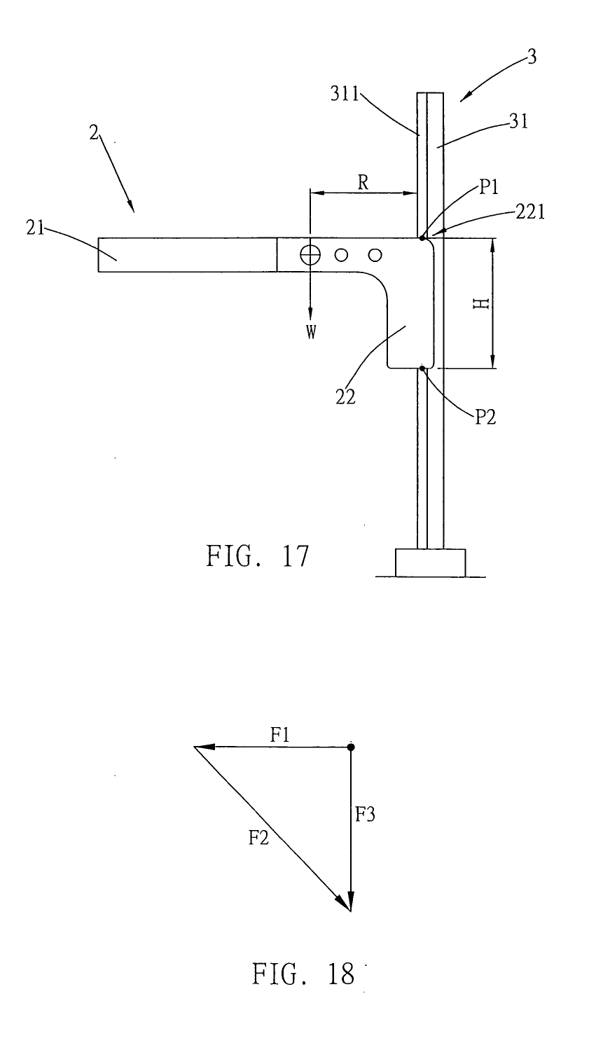

Referring to FIG. 17, which shows the floating support unit 2 structured from the floating support 21 and the mount 22 connected thereto. The floating support unit 2 has a center of gravity W that forms an arm of force R between a point of reaction force P1 or P2 when the mount 22 is positioned on the slide track 31 of the limit device 3. The points of reaction P1, P2 are located on a vertical line of the slide track 31. The slide holes 221 defined in the mount 22 are separated by a height H, and floating displacement of the floating support unit 2 depends on buoyancy effect of the operating liquid 400 on the floating support 21 and a counteractive moment of force resulting from the center of gravity W and the arm of force R.

Referring to FIG. 18, a force from the point of reaction force P1 to the point of reaction force P2 is represented by F3, thereby forming an oblique force F2 between the center of gravity W and the point of reaction force P2. Moreover, because of the arm of force relationship, thus, tension Fl is formed between the point of reaction force P1 and the center of gravity W.

With such a force configuration, if the floating support 12 descends under its own weight, then the tension F1 from the combined force of the component forces F2 and F3 is adequate to form a downward displacement force that is countervailed by friction at the point of reaction force P1. Condition for the downward displacement force to be countervailed is that the points of reaction force P1, P2 must be separated by the height H in order to produce an adequate component force.

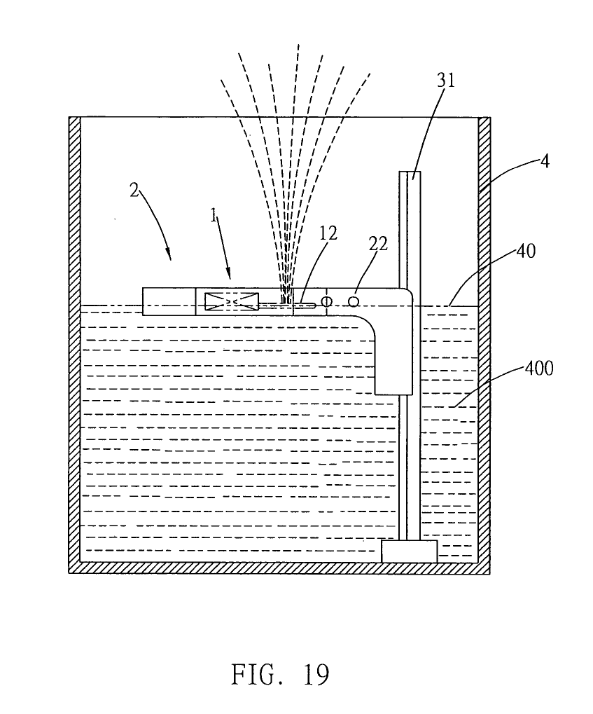

Referring to FIG. 19, the aforementioned structure enables limited vertical displacement of the floating support unit 2 on the slide track 31, and allows the supported excitation device 1 to be effectively positioned on the liquid surface 40 of the operating liquid 400 filled in the container 4. Hence, the floating support unit 2 is able to descend by means of the sliding movement of the mount 22, and further enables the excitation device 1 to maintain a horizontal position on the liquid surface 40.

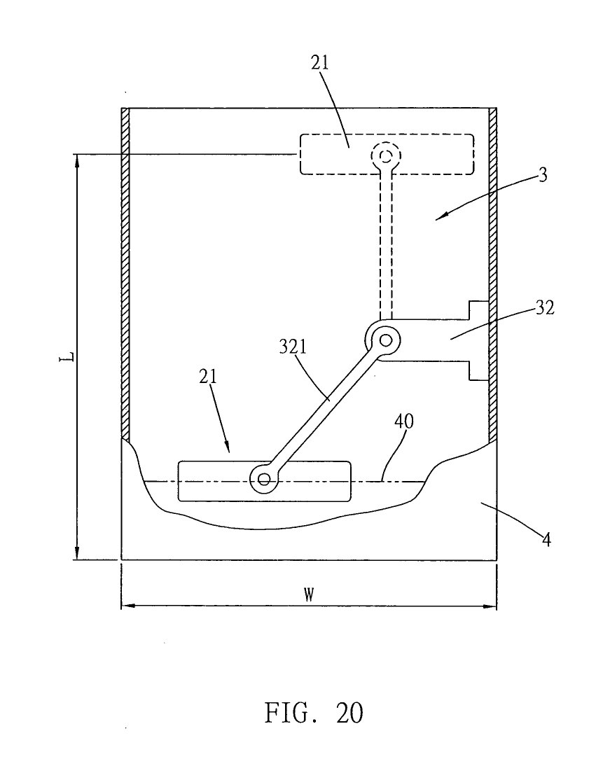

Referring to FIG. 20, which shows the limit device 3 further configured with a pivotal connecting mount 32 joined to one side of the container 4. A swing arm 321 is connected to the pivotal connecting mount 32 using a pin joint method. A free end of the swing arm 321 is pin jointed to the floating support 21, and angular displacement of the floating support 21 can be specified within the range of the swing length and arc length of the swing arm 321. Because height position of the floating support 21 depends on height of the liquid surface 40 on which it floats, thus swing length L of the swing arm 321 is restricted by the height position of the floating support 21.

A connecting method of the swing arm 321 is used to specify angular floating support position of the floating support 21, which is basically to achieve a horizontal disposition on the liquid surface 40.

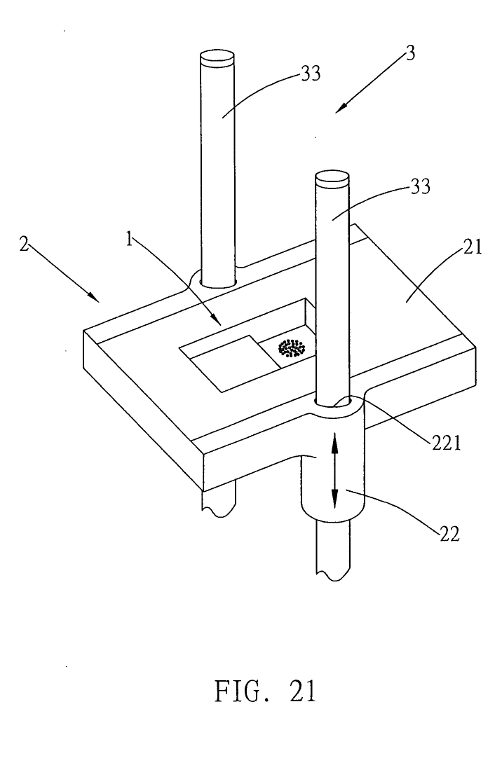

Referring to FIG. 21, which shows the floating support unit 2 confined to the limit device 3 through the mounts 22. The limit device 3 is structured from slide columns 33, an outer periphery of which enable the floating support unit 2 to be disposed and slide thereon through the slide holes 221 of the mount 22, wherein the slide holes have the same shape as that of the slide columns 33. The floating support 21 joined to the floating support unit 2 is thereby able to support the excitation device 1.

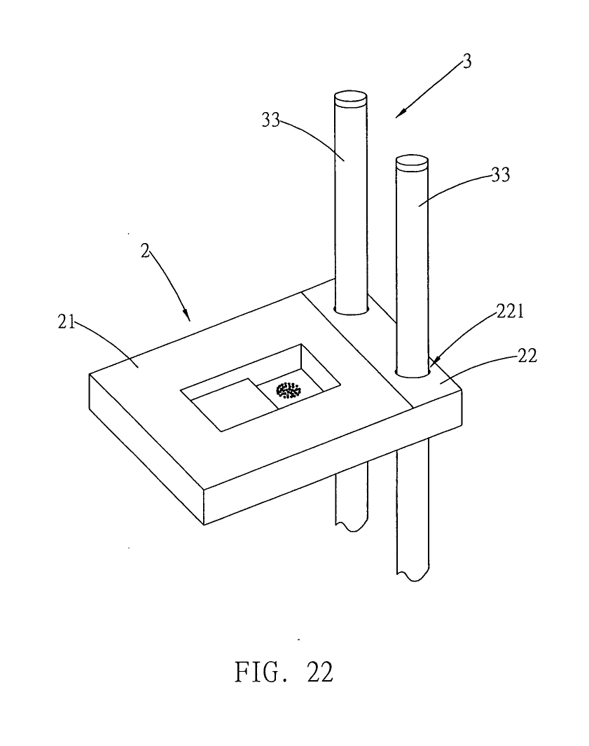

Referring to FIG. 22, which shows the mount 22 joined to one side of the floating support 21 of the floating support unit 2, wherein the mount 22 is disposed and slides on the slide columns 33 through the slide holes 221, thereby supporting the floating support unit 2 using a cantilever method.

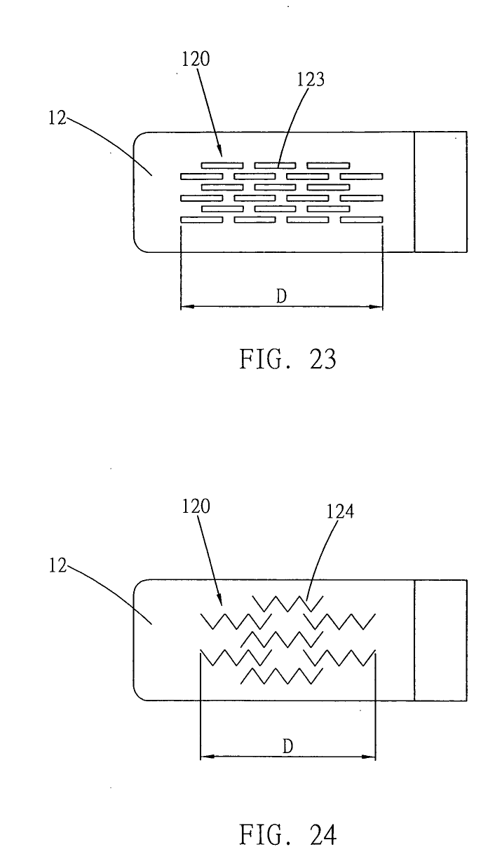

Referring to FIG. 23, which shows the breadth of the vibratable plate 12 defined with the vibratable holes 120, which are narrow linear slots 123 distributed in a staggered arrangement adjacent to each other on the breadth of the vibratable plate 12, the arrangement having a definite front-rear operating length range D.

Because the slots 123 are of narrow linear form, thus, granules equal in width to the slots 123 or granular substances smaller in size can pass through the slots 123, but granules contained in the liquid larger than the width of the slots 123 will be obstructed by the slots 123. However, the slots 123 obstructed by the relatively larger granular substances will not cause complete blockage, but rather form a filtering effect.

Referring to FIG. 24, which shows the vibratable holes formed as waveform slots 124, which are distributed in a staggered arrangement adjacent to each other on the breadth of the vibratable plate 12, the arrangement having the definite front-rear operating length range D.

Referring again to FIG. 5, which shows application of the operating length range D formed from an assembly of the aforementioned slots 123 (124) whereby, after the free end of the vibratable plate 12 is obliquely submerged beneath the liquid surface 40, an intersection point P is formed at any one position within the length range D that enables liquid vibration at the position of the intersection point P of the liquid surface 40, and vibrational energy generated at the free end of the submerged vibratable plate 12 agitates the liquid.

When the vibratable holes 120 are formed as the waveform slots 124, the waveform of the slots 124 can be used to lengthen distance of the slot linear length.

It is of course to be understood that the embodiments described herein are merely illustrative of the principles of the invention and that a wide variety of modifications thereto may be effected by persons skilled in the art without departing from the spirit and scope of the invention as set forth in the following claims.

What is claimed is:

1. A mechanism for the draft of a high frequency atomization device, which has particular application to supporting a cantilever excitation device on the surface of a large amount of operating liquid using a floating support method, thereby enabling a vibratable plate to effectively position and bring into effect quantitative atomization power; the excitation device comprises a block piezoelectric ceramic actuator and the vibratable plate that extends from one side of the actuator and is joined thereto using a cantilever method, and vibratable holes are defined in a breadth of the vibratable plate; the excitation device is joined to a floating support that floats on the surface of the operating liquid; an operating side of a free end of the vibratable plate maintains a definite directed amount of effect on the liquid surface, and is able to acquire comparable load conditions and bring into effect quantitative power.

2. The mechanism for the draft of a high frequency atomization device according to claim 1, wherein a joining surface is used to join together the actuator and the vibratable plate using a soldering method.

3. The mechanism for the draft of a high frequency atomization device according to claim 1, wherein a change-direction member is used to adjust relative horizontal position between the vibratable plate and the actuator.

4. The mechanism for the draft of a high frequency atomization device according to claim 1, wherein a bent portion is used to adjust relative oblique angular relationship between the vibratable plate and the actuator.

5. The mechanism for the draft of a high frequency atomization device according to claim 1, wherein the actuator and the vibratable plate are planar joined, and the planar joined structure is assembled on an oblique side of the floating support in an oblique relationship therewith.

6. The mechanism for the draft of a high frequency atomization device according to claim 1, wherein the floating support is formed with an indentated opening, two sides of which form corners that protect the vibratable plate of the actuator.

7. The mechanism for the draft of a high frequency atomization device according to claim 1, wherein the floating support is a frame-shaped design, an internal through hole of which enables the excitation device to be disposed therein, thereby enabling the vibratable plate to come in close contact with the liquid surface.

8. The mechanism for the draft of a high frequency atomization device according to claim 7, wherein a balance weight is attached to a bottom portion of the frame-shaped floating support.

9. The mechanism for the draft of a high frequency atomization device according to claim 1, wherein symmetrical vibratable plates respectively extend from two opposite sides of the block actuator.

10. The mechanism for the draft of a high frequency atomization device according to claim 9, wherein the change-direction members are used to adjust horizontal position of the two vibratable plates relative to the actuator.

11. The mechanism for the draft of a high frequency atomization device according to claim 9, wherein the bent portions are used to adjust angular relationship of the two vibratable plates relative to the actuator.

12. The mechanism for the draft of a high frequency atomization device according to claim 9, wherein the actuator is assembled in an interior position of the floating support using a beam.

13. The mechanism for the draft of a high frequency atomization device according to claim 1, wherein the block actuator is a square-shaped design.

14. The mechanism for the draft of a high frequency atomization device according to claim 9, wherein the block actuator is a square-shaped design.

15. The mechanism for the draft of a high frequency atomization device according to claim 1, wherein the block actuator is a circular disk-shaped design.

16. The mechanism for the draft of a high frequency atomization device according to claim 9, wherein the block actuator is a circular disk-shaped design.

17. A mechanism for the draft of a high frequency atomization device, which has particular application to supporting a cantilever excitation device on the surface of a large amount of operating liquid using a floating support method, thereby enabling a vibratable plate to effectively position and bring into effect quantitative atomization power; the excitation device comprises a block piezoelectric ceramic actuator and the vibratable plate that extends from one side of the actuator and is joined thereto using a cantilever method; vibratable holes are defined in a breadth of the vibratable plate; and the excitation device is joined to a floating support that floats on the surface of the operating liquid; an operating side of a free end of the vibratable plate maintains a definite directed amount of effect on the liquid surface, and is able to acquire comparable load conditions and bring into effect quantitative power; the floating support is joined to a mount to form a floating support unit that is limited to move within a container by means of a limit device, which limits the floating support unit to vertical displacement.

18. The mechanism for the draft of a high frequency atomization device according to claim 17, wherein the limit device comprises a slide track that uses rails to dispose and slide in slide holes defined in the mount, the slide holes having the same shape as the rails.

19. The mechanism for the draft of a high frequency atomization device according to claim 17, wherein the limit device is configured with a pivotal connecting mount joined to one side of the container, and a swing arm of the pivotal connecting mount is pin jointed to the floating support.

20. The mechanism for the draft of a high frequency atomization device according to claim 17, wherein the limit device comprises slide columns that enable the mount to be disposed and slide thereon through the slide holes defined in the mount, the slide holes having the same shape as the slide columns.

21. The mechanism for the draft of a high frequency atomization device according to claim 17, wherein the limit device is assembled at a side position of the floating support unit.

22. The mechanism for the draft of a high frequency atomization device according to claim 1, wherein the vibratable holes defined in the breadth of the vibratable plate are circular holes.

23. The mechanism for the draft of a high frequency atomization device according to claim 17, wherein the vibratable holes defined in the breadth of the vibratable plate are circular holes.

24. The mechanism for the draft of a high frequency atomization device according to claim 22, wherein a plurality of the circular holes are distributed over any geometrical area.

25. The mechanism for the draft of a high frequency atomization device according to claim 23, wherein a plurality of the circular holes are distributed over any geometrical area.

26. The mechanism for the draft of a high frequency atomization device according to claim 1, wherein the vibratable holes defined in the breadth of the vibratable plate are distributed in a staggered arrangement adjacent to each other, and the vibratable holes are formed as narrow linear slots distributed over a definite operating length range.

27. The mechanism for the draft of a high frequency atomization device according to claim 17, wherein the vibratable holes defined in the breadth of the vibratable plate are distributed in a staggered arrangement adjacent to each other, and the vibratable holes are formed as narrow linear slots distributed over a definite operating length range.

28. The mechanism for the draft of a high frequency atomization device according to claim 26, wherein each of the narrow slots are of straight-line form.

29. The mechanism for the draft of a high frequency atomization device according to claim 27, wherein each of the narrow slots are of straight-line form.

30. The mechanism for the draft of a high frequency atomization device according to claim 26, wherein each of the narrow slots are of waveform.

31. The mechanism for the draft of a high frequency atomization device according to claim 27, wherein each of the narrow slots are of waveform.
